(19) United States
(12) Patent Application Publication
(10) Pub. No.: US 2026/0191545 A1
(43) Pub. Date: Jul. 9, 2026

(54) THROMBUS REMOVAL SYSTEMS AND ASSOCIATED METHODS

(71) Applicant: Shifamed Holdings, LLC, Campbell, CA (US)

(72) Inventor: Tom SAUL, Portland, OR (US)

(21) Appl. No.: 18/868,616

(22) PCT Filed: May 26, 2023
(86) PCT No.: PCT/US2023/067551

§ 371 Date: Nov. 22, 2024

Related U.S. Application Data
(60) Provisional application No. 63/365,376, filed on May 26, 2022.

(51) Int. Cl. A61B 17/22; A61B 17/00; A61B 17/3203

(52) U.S. Cl. CPC A61B 17/22; A61B 17/00234; A61B 17/32037; A61B 2017/00305; A61B 2017/22049; A61B 2017/22055; A61B 2017/22069; A61B 2017/22079; A61B 2217/005

(57) ABSTRACT

The present technology relates to systems and methods for removing a thrombus from a blood vessel of a patient. In some embodiments, the present technology is directed to systems including an elongated catheter having a distal portion configured to be positioned within the blood vessel of the patient, a proximal portion configured to be external to the patient, and a lumen extending therebetween. The system can also include a fluid delivery mechanism coupled with a fluid lumen and configured to apply fluid to at least partially fragment the thrombus, and an aspiration mechanism fluidly coupled to an aspiration lumen and configured to aspirate the fragmented thrombus.

### CLAIM OF PRIORITY AND RELATED APPLICATION

This application claims the benefit of U.S. Provisional Application No. 63/365,376, filed May 26, 2022, which is herein incorporated by reference in its entirety for all purposes. This application is related to International Application No. PCT/US2021/020915, filed Mar. 4, 2021, and U.S. Application No. 63/190,784, filed May 19, 2021, the disclosures of which are incorporated by reference herein.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present technology generally relates to medical devices and, in particular, to systems including aspiration and fluid delivery mechanisms and associated methods for removing a thrombus from a mammalian blood vessel.

### BACKGROUND

Thrombotic material may lead to a blockage in fluid flow within the vasculature of a mammal. Such blockages may occur in varied regions within the body, such as within the pulmonary system, peripheral vasculature, deep vasculature, or brain. Pulmonary embolisms typically arise when a thrombus originating from another part of the body (e.g., a vein in the pelvis or leg) becomes dislodged and travels to the lungs. Anticoagulation therapy is the current standard of care for treating pulmonary embolisms, but may not be effective in some patients. Additionally, conventional devices for removing thrombotic material may not be capable of navigating the vascular anatomy of the lungs, may not be effective in removing thrombotic material, and/or may lack the ability to provide sensor data or other feedback to the clinician during the thrombectomy procedure.

### SUMMARY

A medical system is provided, comprising: a compliant shaft; a guidewire system coupled to the compliant shaft, the guidewire system including: a lumen configured to receive a guidewire; a plurality of inflatable members disposed along the lumen, the plurality of inflatable members being configured to be inflated sequentially to engage with the guidewire and decrease a length of the lumen to cause the guidewire system and compliant shaft to advance along the guidewire.

In one aspect, the plurality of inflatable members comprise inflatable walls of a plurality of inflation lumens.

In another aspect, the plurality of inflatable members are configured to be inflated sequentially from a distal-most inflatable member to a proximal-most inflatable member to decrease the length of the lumen.

In one aspect, inflation of the distal-most inflatable member is configured to anchor the guidewire system to the guidewire.

In some aspects, the plurality of inflatable members are annular balloons. In another aspect, the plurality of inflatable members are H-shaped annular balloons.

In some aspects, the compliant shaft comprises a thrombectomy device.

In another aspect, the system includes an expandable funnel disposed near a proximal end of the compliant shaft. In another aspect, the system comprises an aspiration lumen disposed in the compliant shaft. In some aspects, the system includes one or more fluid ports disposed in the compliant shaft and configured to direct two or more fluids streams into the expandable funnel.

A thrombus removal device is provided, comprising: an elongate catheter shaft configured to be positioned withing a blood vessel of a patient; an expandable distal portion coupled to the elongate catheter shaft; and a plurality of inflatable members disposed on the elongate catheter shaft, the plurality of inflatable members being configured to be inflated sequentially to decrease a length of the elongate catheter and cause the thrombus removal device to advance within the blood vessel.

In another aspect, the device includes an expandable distal portion coupled to the elongate catheter shaft.

In some aspects, the device includes a plurality of fluid ports disposed in the elongate catheter shaft or the expandable distal portion, the plurality of fluid ports being configured to direct a plurality of fluid streams into the expandable distal portion.

In other aspects, the plurality of inflatable members are disposed on an exterior surface of the elongate catheter shaft. In some aspects, the plurality of inflatable members are displaced axially along the elongate catheter shaft.

In another aspect, the plurality of inflatable members are configured to be inflated sequentially from a distal-most inflatable member to a proximal-most inflatable member to decrease the length of the elongate catheter shaft.

In one aspect, inflation of the distal-most inflatable member is configured to anchor the elongated catheter device within the blood vessel.

In another aspect, the plurality of inflatable members are annular balloons.

In some aspects, the plurality of inflatable members are H-shaped annular balloons.

In other aspects, the plurality of inflatable members are configured to engage with a wall of the blood vessel when inflated.

In one aspect, the plurality of inflatable members are fluidly coupled to an inflation system with one or more inflation lumens in the elongate catheter shaft.

A guidewire system is provided, comprising: a compliant shaft; a lumen disposed in the compliant shaft and configured to receive a guidewire; a plurality of inflatable members disposed in the compliant shaft, the plurality of inflatable members being configured to be inflated sequentially to engage with the guidewire and decrease a length of the compliant shaft to cause the guidewire system to advance along the guidewire disposed within the lumen.

In some aspects, the plurality of inflatable members comprise inflatable walls of a plurality of inflation lumens.

In another aspect, the plurality of inflatable members are configured to be inflated sequentially from a distal-most inflatable member to a proximal-most inflatable member to decrease the length of the compliant shaft.

In some aspects, inflation of the distal-most inflatable member is configured to anchor the guidewire system to the guidewire.

In another aspect, the plurality of inflatable members are annular balloons. In some aspects, the plurality of inflatable members are H-shaped annular balloons.

In one aspect, the device further includes a medical catheter device coupled to the guidewire system.

In some embodiments, the medical catheter device comprises a thrombectomy device.

In another aspect, the medical catheter device is configured to advance along the guidewire with the guidewire system.

A method of advancing a guidewire system within a lumen of a patient is provided, comprising: inserting a guidewire into a blood vessel; placing the guidewire system over the guidewire; sequentially inflating a plurality of inflatable members disposed along a length of the guidewire system to engage the guidewire and decrease a length of the guidewire system; and sequentially deflating the plurality of inflatable members to increase a length of the guidewire system to advance the guidewire system over the guidewire.

In some aspects, sequentially inflating the plurality of inflatable members further comprises sequentially inflating from a distal-most inflatable member to proximal-most inflatable member.

In another aspect, sequentially deflating the plurality of inflatable members further comprises deflating from a distal-most inflatable member to a proximal-most inflatable member.

A method of advancing a medical device within a lumen of a patient is provided, comprising: inserting a catheter into a blood vessel; sequentially inflating a plurality of inflatable members disposed along a length of the catheter to engage the blood vessel and decrease a length of the catheter; and sequentially deflating the plurality of inflatable members to increase a length of the catheter to advance the medical device along the blood vessel.

In some aspects, sequentially inflating the plurality of inflatable members further comprises sequentially inflating from a distal-most inflatable member to proximal-most inflatable member.

In another aspect, sequentially deflating the plurality of inflatable members further comprises deflating from a distal-most inflatable member to a proximal-most inflatable member.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1-1A illustrate various views of a portion of a thrombus removal system including a distal portion of an elongated catheter configured in accordance with an embodiment of the present technology.

FIGS. 2A-2G illustrate one embodiment of an advancement mechanism of a thrombus removal device.

FIGS. 3A-3C illustrate one embodiment of a guidewire system of a thrombus removal device.

FIG. 4 is a method of advancing a guidewire system along a guidewire.

FIG. 5 is a method of advancing a catheter along a body lumen.

### DETAILED DESCRIPTION

The present technology is generally directed to thrombus removal systems and associated methods. A system configured in accordance with an embodiment of the present technology can include, for example, an elongated catheter having a distal portion configured to be positioned within a blood vessel of the patient, a proximal portion configured to be external to the patient, a fluid delivery mechanism configured to fragment the thrombus with pressurized fluid, an aspiration mechanism configured to aspirate the fragments of the thrombus, and one or more lumens extending at least partially from the proximal portion to the distal portion ..

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples but are not described in detail with respect to the figures.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “generally,” “approximately,” and “about” are used herein to mean the stated value plus or minus 10%.

Although some embodiments herein are described in terms of thrombus removal, it will be appreciated that the present technology can be used and/or modified to remove other types of emboli that may occlude a blood vessel, such as fat, tissue, or a foreign substance. Additionally, although some embodiments herein are described in the context of thrombus removal from a pulmonary artery (e.g., pulmonary embolectomy), the technology may be applied to removal of thrombi and/or emboli from other portions of the vasculature (e.g., in neurovascular, coronary, or peripheral applications). Moreover, although some embodiments are discussed in terms of maceration of a thrombus with a fluid, the present technology can be adapted for use with other techniques for breaking up a thrombus into smaller fragments or particles (e.g., ultrasonic, mechanical, enzymatic, etc.).

The headings provided herein are for convenience only and do not interpret the scope or meaning of the claimed present technology.

### Systems for Thrombus Removal

As provided above, the present technology is generally directed to thrombus removal systems. Such systems include an elongated catheter having a distal portion positionable within a blood vessel of the patient (e.g., an artery or vein), a proximal portion positionable outside the patient's body, a fluid delivery mechanism configured to fragment the thrombus with pressurized fluid, an aspiration mechanism configured to aspirate the fragments of the thrombus, and one or more lumens extending at least partially from the proximal portion to the distal portion. In some embodiments, the systems herein are configured to engage a thrombus in a patient's blood vessel, break the thrombus into small fragments, and aspirate the fragments out of the patient's body. The pressurized fluid streams (e.g., jets) function to cut or macerate thrombus, before, during, and/or after at least a portion of the thrombus has entered the aspiration lumen or a funnel of the system.

Fragmentation helps to prevent clogging of the aspiration lumen and allows the thrombus removal system to macerate large, firm clot that otherwise could not be aspirated. As used herein, “thrombus” and “embolism” are used somewhat interchangeably in various respects. It should be appreciated that while the description may refer to removal of “thrombus,” this should be understood to encompass removal of thrombus fragments and other emboli as provided herein.

According to embodiments of the present technology, a fluid delivery mechanism can provide a plurality of fluid streams (e.g., jets) to fluid apertures of the thrombus removal system for macerating, cutting, fragmenting, pulverizing and/or urging thrombus to be removed from a proximal portion of the thrombus removal system. The thrombus removal system can include an aspiration lumen extending at least partially from the proximal portion to the distal portion of the thrombus removal system that is adapted for fluid communication with an aspiration pump (e.g., vacuum source). In operation, the aspiration pump may generate a volume of lower pressure within the aspiration lumen near the proximal portion of the thrombus removal system, urging aspiration of thrombus from the distal portion. Additional techniques for removing clots can also be provided, including simple suction, cages/capture, suction and jets, or any other technique known in the art.

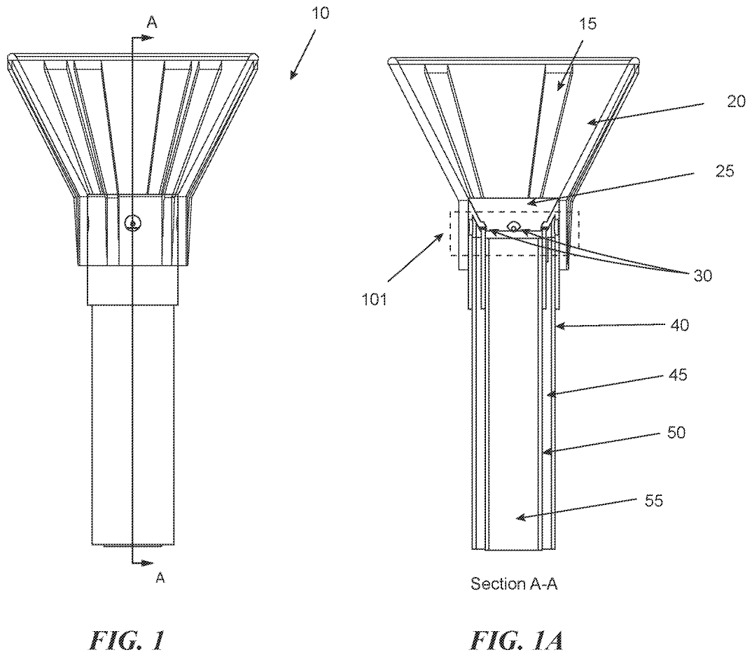

FIG. 1 illustrates a distal portion 10 of a thrombus removal system according to an embodiment of the present technology. FIG. 1A Section A-A illustrates an elevation sectional view of the distal portion. The example section A-A in FIG. 1A depicts a funnel 20 that is positioned at the distal end of the distal portion 10, the funnel adapted to engage with thrombus and/or a tissue (e.g., vessel) wall to aid in thrombus fragmentation and/or removal. The example section A-A in FIG. 1A depicts a double walled thrombus removal device construction having an outer wall/tube 40 and an inner wall/tube 50. An aspiration lumen 55 is formed by the inner wall 50 and is centrally located. A generally annular volume forms at least one fluid lumen 45 between the outer wall 40 and the inner wall 50. The fluid lumen 45 is adapted for fluid communication with a fluid delivery mechanism. One or more apertures (e.g., nozzles, orifices, or ports) 30 are positioned in the thrombus removal system to be in fluid communication with the fluid lumen 45 and an irrigation manifold 25. In operation, the ports 30 are adapted to direct (e.g., pressurized) fluid toward thrombus that is engaged with the distal portion 10 of the thrombus removal system. In some embodiments, the manifold can be positioned proximal to the funnel. In other embodiments, the manifold can be integrated into the funnel.

In some embodiments, one or more of the lumens formed in the thrombus removal device can be adapted to carry an inflation medium for inflating/deflating one or more inflatable members. In some examples, the fluid lumen 45 can be used for inflation/deflation. In another embodiment, a separate inflation lumen separate from the fluid lumen can be formed between the inner and outer walls of the device. In this embodiment, inflation of the inflatable members can be accomplished with the inflation lumen, and fluid delivery via jets or ports in the device can be accomplished with the fluid lumen. The inflation lumen can be fluidly coupled to an inflation system, for example.

In some embodiments described herein, the outer wall/tube 40 and an inner wall/tube 50 can be formed of a substantially flexible or compliant material. For example, the shaft or inner/outer walls of the thrombus removal device can be formed from a polymer, a vinyl, a silicon, a plastic, or any other flexible materials routinely used in the formation of flexible catheters or vessel/lumen access devices, as known in the art. Forming the shaft or walls of the thrombus removal system with a flexible/compliant material allows for navigation of tortuous or bendy lumens, such as the blood vessel network within a human brain.

The thrombus removal system can be sized and configured to access and remove thrombi in various locations or vessels within a patient's body. It should be understood that while the dimensions of the system may vary depending on the target location, generally the same features and components described herein will be implemented in the thrombus removal system regardless of the application. For example, a thrombus removal system configured to remove pulmonary embolism (PE) from a patient may have an outer wall/tube with a size of approximately 11-13 Fr, or preferably 12 Fr, and an inner wall/tube with a size of 7-9 Fr, or preferably 8 Fr. A deep vein thrombosis (DVT) device, on the other hand, may have an outer wall/tube with a size of approximately 9-11 Fr, or preferably 10 Fr, and an inner wall/tube with a size of 6-9 Fr, or preferably 7.5 Fr. Applications are further provided for ischemic stroke and peripheral embolism applications.

In some embodiments, the ports are formed to direct the fluid flow along a selected path. In some embodiments, at least two ports are arranged to produce (e.g., respective) fluid streams that intersect at an intersection region of the thrombus removal system. An intersection region can be a region of increased fluid momentum and/or energy transfer, which increase is with respect to individual fluid streams that are not directed to combine at the intersection. The increased fluid momentum and/or energy transfer at an intersection may advantageously fragment thrombus more efficiently and/or quickly. In some embodiments, an intersection region can be formed from at least 2, at least 3, at least 4, at least 5, at least 6, at least 7, at least 8, at least 9, or at least 10 fluid streams. An intersection region can be generally near a central axis of the thrombus removal system, or away from the central axis. In some embodiments, at least two intersection regions are formed. In some embodiments, one or more are arranged to direct a fluid stream along an oblique angle with respect to the central axis of the thrombus removal system. An operating pressure of the fluid delivery mechanism may be selected to approach a targeted fluid velocity for a fluid stream that is delivered from a port. In some embodiments, at least two ports are adapted to delivery respective fluid streams at different fluid velocities, for a given pressure of the fluid delivery mechanism. In some embodiments, at least two ports are adapted to delivery respective fluid streams at the substantially the same fluid velocities, for a given pressure of the fluid delivery mechanism. In some embodiments, angular momentum is imparted to a thrombus by application of a) at least one fluid stream that is directed at an oblique angle from a port, and/or b) at least two fluid streams that have different fluid velocities. Advantageously, angular momentum produced in a thrombus may impart a (e.g., centrifugal) force that assists in fragmentation and removal of the thrombus. Advantageously, an increased cross-sectional area of the fluid lumen reduces a required operating pressure of the fluid delivery mechanism to achieve a targeted fluid velocity of the fluid streams.

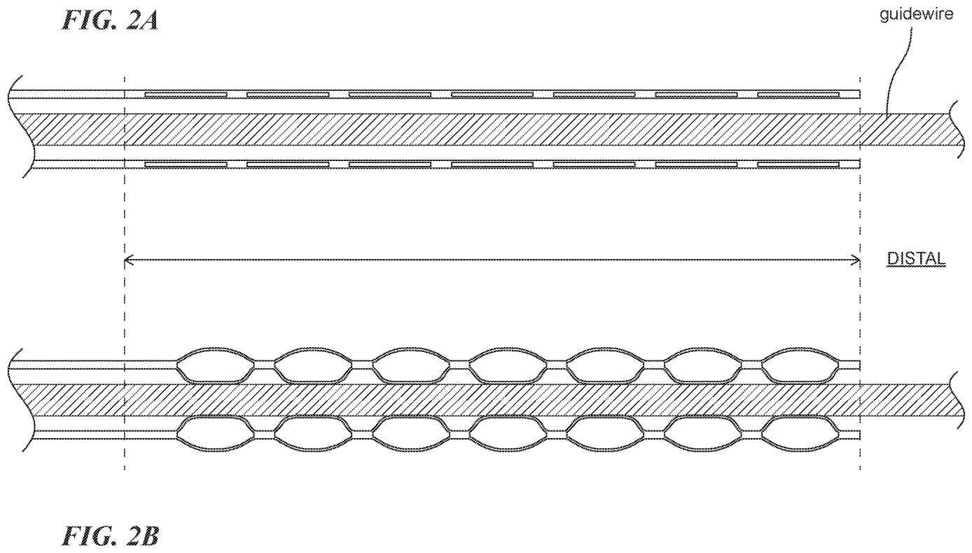

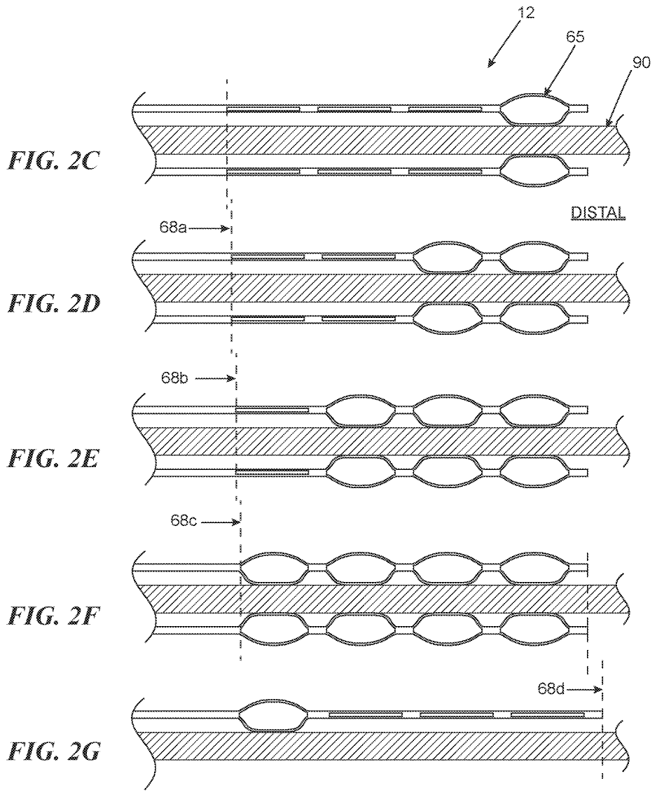

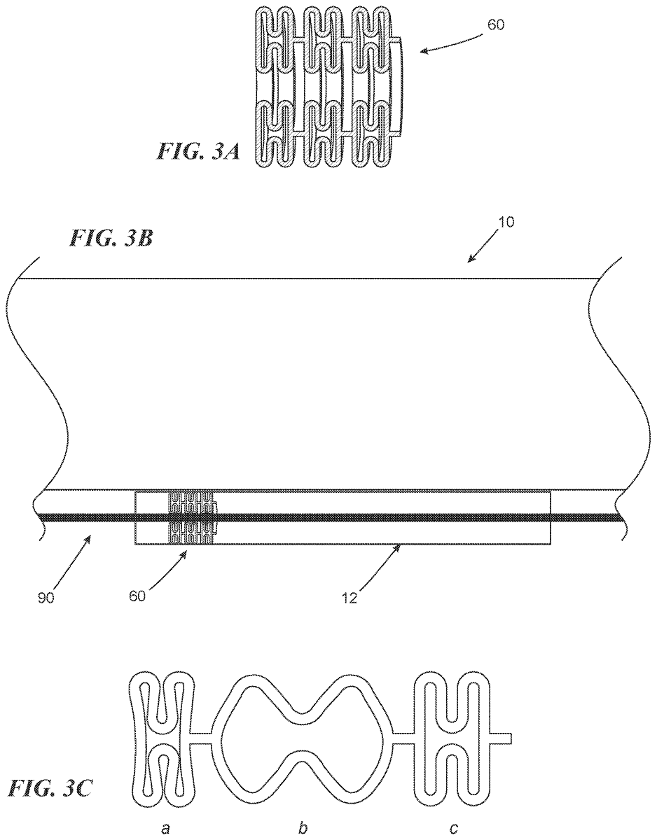

In one embodiment, referring to FIGS. 2A-2G, an advancement mechanism 60 is disposed on or within a guidewire system 12 and configured to move a medical device, such as a thrombus removal system, along the guidewire system. While this disclosure refers generally to a thrombus removal device, it should be understood that any catheter based or minimally invasive medical device can be used with the advancement mechanism and guidewire system described herein. The guidewire system can provide the advancement mechanism 60 around a guidewire lumen 85, where the advancement mechanism is configured to engage with a guidewire 90. The advancement mechanism is configured to engage with the guidewire to move the guidewire system distally and/or proximally along the guidewire by sequentially inflating and deflating a plurality of inflatable members. Referring to FIG. 2A, the guidewire system 12 can include inner and outer walls and a space between the inner and outer walls that includes one or more inflation lumens 70, the inflation lumens being fluidly coupled to the plurality of inflatable members 65. In some embodiments, the inflatable members herein may be deformable walls of the inflation lumens. FIG. 2A shows the inflation lumens 70 when the plurality of inflatable members 65 are deflated, and FIG. 2B shows the device with the inflatable members 65 inflated so as to expand outwards radially from the shaft or distal portion of the system.

As shown in FIGS. 2A-2B, inflating the inflatable members 65 also causes the length of the advancement mechanism to shorten longitudinally. For example, it can be seen that the distal ends of the advancement mechanism are aligned in FIGS. 2A-2B, but the proximal end of the advancement mechanism in FIG. 2B is shorter (or moved distally) with respect to the proximal end of the advancement mechanism in FIG. 2A. Stated another way, with the inflatable members fully deflated (e.g., FIG. 2A), the advancement mechanism section has a length that is longer than a length of the advancement mechanism section of the distal portion in FIG. 2B, in which the inflatable members are inflated.

FIGS. 2C-2F illustrate how the advancement mechanism 60 can be used to advance the guidewire system 12 along the guidewire 90. As shown in FIG. 2C, the distal most inflatable member 65 is inflated, while the remaining inflation lumens 70 remain deflated. This can cause the advancement mechanism 60 to engage with or grab onto the guidewire 90. This also causes the advancement mechanism section to shorten relative to the uninflated configuration of FIG. 2A.

Next, referring to FIG. 2D, the second distal-most inflatable member 65 is inflated, causing the advancement mechanism section to shorten relative to the configuration of FIG. 2C. The amount of shortening is shown with reference line 68a. As subsequent inflatable members are inflated in FIGS. 2E-2F, the advancement mechanism section continues to shorten by lengths 68b and 68c, respectively, as more inflatable members 65 are inflated. The distal end of the advancement mechanism section remains anchored or tethered to the guidewire, as shown.

As will be understood by a skilled artisan, since the distal most inflatable member anchors the guidewire system to the guidewire, each subsequent inflation of an inflatable member shortens the length of the guidewire system in a distal direction (e.g., towards the anchored distal-most inflatable member), thereby advancing the proximal portion of the guidewire system distally towards the distal portion of the guidewire system. At this point, referring to FIG. 2G the inflatable members can be deflated in the same fashion (e.g., deflating distal-most to proximal-most), causing the advancement mechanism section of the guidewire system to increase in length. Since the distal-most inflatable member is the first to be deflated, it is able to expand in length by a distance 68d, anchored by the other inflatable members against the guidewire, effectively causing the distal portion to “pull itself” along the guidewire system. This process can then be repeated until the guidewire system is in the desired target location with a lumen or blood vessel.

With that sequence now described it can also be understood how the device can be retracted in the proximal direction within a vessel or lumen, essentially replicating the inflation/deflation process in reverse order (e.g., inflating proximal to distal, then deflating proximal to distal). With this technique, the flexibility and compliance of the guidewire system can be used to navigate even the most tortuous sections of blood vessels or lumens, such as blood vessels within a brain of a patient.

In another embodiment, referring to FIGS. 3A-3C, the guidewire system 12 can be attached to a medical device 10, such as a thrombus removal device or any other minimally invasive or catheter-based device. As described above, the guidewire system can provide the advancement mechanism 60 around a guidewire lumen, where the advancement mechanism is configured to engage with a guidewire 90 using similar principles to those described above. In use, the guidewire 90 can be advanced through the guidewire lumen 85 and advanced to a target tissue location, such as within a vessel or lumen. When the guidewire is in place, then the inflatable members 65 can be inflated and deflated similar to as described above, causing the guidewire system 12 to “grab” onto the guidewire with the inflatable members to pull the guidewire system along the guidewire towards the target tissue location. The sequential inflation/deflation of the inflatable members can be reversed to move the guidewire system in the opposite direction.

It is contemplated that the same principles described above with the guidewire system can also be used to cause a catheter-based or minimally invasive medical device to crawl or inch along a vessel wall. Instead of the inflatable members grabbing onto a guidewire, as described above, the inflatable members can instead grab onto a vessel wall itself. The same principles of inflation and deflation to move the device along the vessel wall would apply. For example, the thrombus removal device of FIGS. 1 and 1A could include inflatable members 65 and inflation lumens 70 along the outer wall 40 of the device. With the funnel 20 collapsed or deployed, the inflatable members could be inflated as described above to engage with a vessel wall instead of with a guidewire. The sequential inflation and deflation of the inflatable members could be used to advance or retract the thrombus removal device along the vessel wall without the need for a guidewire.

Many different inflatable member designs are considered. In some embodiments, the inflatable members can be annular or ring balloons that extend around a periphery (or interior) of a thrombus removal device or a guidewire lumen. In another embodiment, referring to FIGS. 3A and 3C, the advancement mechanism 60 can include a plurality of “H” shaped annular balloons, as illustrated. FIG. 3A shows the balloons in a deflated or collapsed state. In this deflated state, the annular balloons comprise their shortest axial length. FIG. 3C shows three annular balloons a, b, and c. Balloon a is fully deflated, balloon b is inflated, and balloon c is partially inflated. It can be see that balloon b has the longest axial length, followed by balloon c. Balloon a has the shortest axial length.

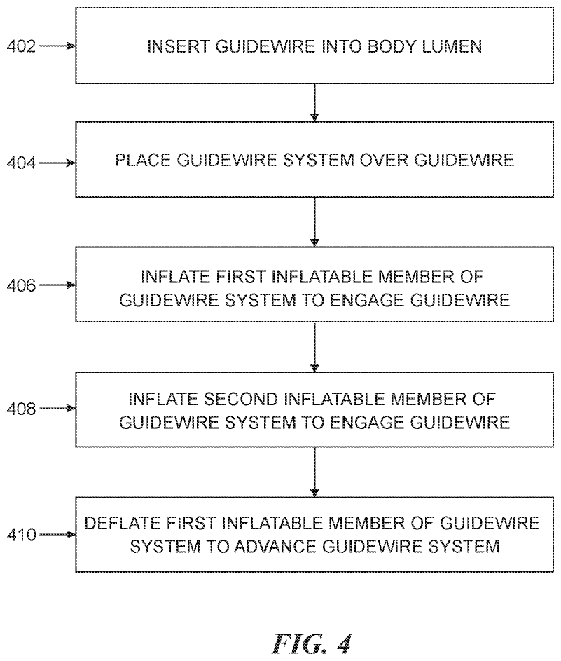

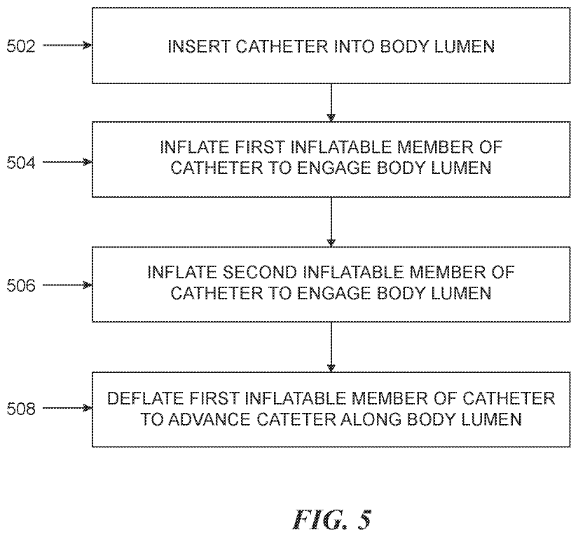

FIGS. 4 and 5 show flowcharts describing methods of advancing a guidewire system or catheter along a body lumen of a patient, such as along a blood vessel.

Referring to FIG. 4, at step 402, a guidewire can be inserted into a body lumen of a patient. The body lumen can comprise, for example, a blood vessel.

At step 404, the guidewire system can be placed over the guidewire. The guidewire system can comprise, for example, the guidewire system 12 of FIGS. 2A-2G or 3B.

At step 406, a first inflatable member of the guidewire system can be inflated to engage the guidewire. As described above, in some embodiments, the first inflatable member comprises a distal-most inflatable member of the guidewire system. Inflation of the inflatable members can cause the guidewire system to shorten axially, as described in FIGS. 2A-2G and 3A-3C.

At step 408, a second inflatable member of the guidewire system can be inflated to engage the guidewire. In some embodiments, the second inflatable member can be proximally located on the guidewire system relative to the first inflatable member. Inflation of the inflatable members can cause the guidewire system to shorten axially.

At step 410, the first inflatable member of the guidewire system can be deflated to advance the guidewire system along the guidewire. In some embodiments, multiple inflatable members are inflated between steps 408 and 410. For example, referring to the embodiment of FIGS. 2C-2G, one, two, three, four, or more inflatable members can be inflated sequentially from a distal-most portion of the guidewire system to a proximal-most portion of the guidewire system.

Any number of the inflatable members can be inflated prior to deflating the first inflatable member in step 410. In one embodiment, all inflatable members are inflated sequentially from distal-most to proximal most, and then starting at step 410, all inflatable members are deflated sequentially from distal-most to proximal most. Inflation of the inflatable members causes them to engage with the guidewire and shorten an axial length of the guidewire system, and deflation of the inflatable members causes them to disengage from the guidewire and increase an axial length of the guidewire system, causing the guidewire system to advance or move along the guidewire. The guidewire is generally held in place within the body lumen while the guidewire system moves along the guidewire.

FIG. 5 is a method that is similar to the one described above in FIG. 4. However, the method of FIG. 5 does not require the use of a guidewire, but instead uses inflatable members to move a catheter device along a body lumen by directly engaging with the body lumen wall(s).

Referring to FIG. 5, at step 502, a catheter can be inserted into a body lumen of a patient. The body lumen can comprise, for example, a blood vessel.

At step 504, a first inflatable member of the catheter can be inflated to engage the body lumen wall(s). As described above, in some embodiments, the first inflatable member comprises a distal-most inflatable member of the catheter. Inflation of the inflatable members can cause the catheter to shorten axially, as described in FIGS. 2A-2G and 3A-3C.

At step 506, a second inflatable member of the catheter can be inflated to engage the body lumen wall(s). In some embodiments, the second inflatable member can be proximally located on the catheter relative to the first inflatable member. Inflation of the inflatable members can cause the catheter to shorten axially.

At step 508, the first inflatable member of the catheter can be deflated to advance the catheter along the body lumen. In some embodiments, multiple inflatable members are inflated between steps 506 and 508. For example, referring to the embodiment of FIGS. 2C-2G, one, two, three, four, or more inflatable members can be inflated sequentially from a distal-most portion of the guidewire system to a proximal-most portion of the guidewire system. Any number of the inflatable members can be inflated prior to deflating the first inflatable member in step 508. In one embodiment, all inflatable members are inflated sequentially from distal-most to proximal most, and then starting at step 508, all inflatable members are deflated sequentially from distal-most to proximal most. Inflation of the inflatable members causes them to engage with the body lumen wall(s) and shorten an axial length of the catheter, and deflation of the inflatable members causes them to disengage from the body lumen wall(s) and increase an axial length of the catheter, causing the catheter to advance or move along the body lumen wall(s).

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A medical system, comprising:
a compliant shaft;
a guidewire system coupled to the compliant shaft, the guidewire system including:
a lumen configured to receive a guidewire;
a plurality of inflatable members disposed along the lumen, the plurality of inflatable members being configured to be inflated sequentially to engage with the guidewire and decrease a length of the lumen to cause the guidewire system and compliant shaft to advance along the guidewire.

2. The system of claim 1 wherein the plurality of inflatable members comprise inflatable walls of a plurality of inflation lumens.

3. The system of claim 1, wherein the plurality of inflatable members are configured to be inflated sequentially from a distal-most inflatable member to a proximal-most inflatable member to decrease the length of the lumen.

4. The system of claim 3, wherein inflation of the distal-most inflatable member is configured to anchor the guidewire system to the guidewire.

5. The system of claim 1, wherein the plurality of inflatable members are annular balloons.

6. The system of claim 1, wherein the plurality of inflatable members are H-shaped annular balloons.

7. The system of claim 1, wherein the compliant shaft comprises a thrombectomy device.

8. The system of claim 1, further comprising an expandable funnel disposed near a proximal end of the compliant shaft.

9. The system of claim 8, further comprising an aspiration lumen disposed in the compliant shaft.

10. The system of claim 9, further comprising one or more fluid ports disposed in the compliant shaft and configured to direct two or more fluids streams into the expandable funnel.

11. A thrombus removal device, comprising:
an elongate catheter shaft configured to be positioned withing a blood vessel of a patient;
an expandable distal portion coupled to the elongate catheter shaft;
a plurality of inflatable members disposed on the elongate catheter shaft, the plurality of inflatable members being configured to be inflated sequentially to decrease a length of the elongate catheter and cause the thrombus removal device to advance within the blood vessel.

12. The thrombus removal device of claim 11, further comprising an expandable distal portion coupled to the elongate catheter shaft.

13. The thrombus removal device of claim 12, further comprising a plurality of fluid ports disposed in the elongate catheter shaft or the expandable distal portion, the plurality of fluid ports being configured to direct a plurality of fluid streams into the expandable distal portion.

14. The thrombus removal device of claim 11, wherein the plurality of inflatable members are disposed on an exterior surface of the elongate catheter shaft.

15. The thrombus removal device of claim 11, wherein the plurality of inflatable members are displaced axially along the elongate catheter shaft.

16. The thrombus removal device of claim 11, wherein the plurality of inflatable members are configured to be inflated sequentially from a distal-most inflatable member to a proximal-most inflatable member to decrease the length of the elongate catheter shaft.

17. The thrombus removal device of claim 16, wherein inflation of the distal-most inflatable member is configured to anchor the elongated catheter device within the blood vessel.

18. The thrombus removal device of claim 11, wherein the plurality of inflatable members are annular balloons.

19. The thrombus removal device of claim 11, wherein the plurality of inflatable members are H-shaped annular balloons.

20. The thrombus removal device of claim 11, wherein the plurality of inflatable members are configured to engage with a wall of the blood vessel when inflated.

21. The thrombus removal device of claim 11, wherein the plurality of inflatable members are fluidly coupled to an inflation system with one or more inflation lumens in the elongate catheter shaft.

22.-36. (canceled)
